(19) United States
(12) Patent Application Publication
(10) Pub. No.: US 2010/0123076 A1
(43) Pub. Date: May 20, 2010

(54) Method for experimentally optic transmitting information through an optic nerve

(76) Inventor: Victor V. Sevastyanov, Yoshkar-Ola (RU)

Correspondence Address: RICHARD L. MILLER, 12 PARKSIDE DRIVE, DIX HILLS, NY, 11746, US

(21) Appl. No.: 12/313,286

(22) Filed: Nov. 19, 2008

(51) Int. Cl. G01N 21/64; A61B 16/00

(52) U.S. Cl. 250/304; 250/459.1

(57) ABSTRACT

A method for experimentally optic transmitting information through an optic nerve. The method includes the steps of projecting a laser beam through the pupil, the vitreous body, the ganglion cells of the retina and the afferent nerve fibers to the beginning of the optic chiasm; observing visible luminescence of the eyeball and afferent nerve fibers to the beginning of the optic chiasm with holographic effects; transecting the optic nerve at the beginning of the optic chiasm in order to check the propagation of a laser beam along the efferent fibers of the optic nerve; protecting the eyeball with a metal screen; fixing the metal screen at the exit of the optic nerve from the eyeball, directing the laser beam at the butt end of the optic nerve, observing visible luminescence of the optic nerve with holographic effect; projecting the laser beam in the pupil, and observing visible luminescence of the pupil.

### BACKGROUND OF THE INVENTION

A. Field of the Invention

The embodiments of the present invention relate to a method for transmitting information, and more particularly, the embodiments of the present invention relate to a method for experimentally optic transmitting information through an optic nerve.

B. Description of the Prior Art

(1) General

It is known that the information received by the retina is transferred through to optic nerve to the brain. The optic nerve is formed by the axons of retinal ganglion cells. The optic nerve of an adult has 1.1 million fibers that transfer electric impulses to the brain. One of the serious reasons breaking down the transfer of information through the optic nerve is its atrophy. Optic nerve atrophy is the result of various pathological precesses. It can occur as a consequence of inflammation, degenerative changes, edema, compression, and damage to the optic nerve.

H. A. Quigley and D. R. Anderson studied clinical and microscopic appearances of the optic nerve head in squirrel monkeys with optic nerve degeneration. Explaining pathogenesis of the acquired optic nerve pallor, they postulated that the transparent nerve fibers act as fiberoptic pathways in conducting light.

The light diffuses among adjacent columns of glial cells and capillaries and acquires the pink color of capillaries. The axon bundles of an atrophic optic disc have been destroyed, and the remaining astrocytes are arranged at angles to the entering light. Thus, little light passes into the disc substance to traverse the capillaries. The light reflected from opaque glial cells does not pass through capillaries, remains white, and the optic disc appears pale. In some areas, loss of tissue allows light to pass directly to the opaque scleral lamina, and this adds to the white color of the disc.¹ ¹Quigley H. A., Anderson D. R., The historical Basis of Optic Disc Pallor. Am. J. Ophthalmol, 1977, 83, pp 709-717.

The optic nerve atrophy is one of the severe conditions that entail considerable visual impairment. The treatment of optic nerve atrophy is usually ineffective.

In order to transfer information from cell to cell, neurons use electrical and chemical signals. J. G. Nicholls et al. describe the consecutive steps of information transfer. The progression can be followed step by step as light falls on the photoreceptors to generate electrical signals, which then influence bipolar cells. From bipolar cells, signals are conveyed to the ganglion cells, and from there onward to higher centers in the brain that give rise to ones perception of the outside world.

Nicholls refers action potentials to the second major category of electrical signals. Action potentials are initiated by localized graded potentials. Unlike local potentials, they propagate rapidly over long distances, for example, from the eye to the higher centers along ganglion cell axons in the optic nerve. It should be noted that outputs from more than 100 million receptors converge to provide input to 1 million ganglion cells, the axons of which make up the optic nerve.

There exists some non-invasive techniques for recording neuronal activity, Magnetic resonance imaging (MRI) is one of them. MRI allows one to determine which regions of the human, awake brain are active when stimuli are presented. Overall measures of the averaged activity of the eye and brain are provided by the electroretinogram and electroencephalogram. They are mainly used to diagnose disorders of function.² ²John G. Nicholls et al., From Neuron to Brain, 4th ed. 2001, Sinauer Associates, Inc.

For over 20 years, the applicant of the embodiments of the present invention has been studying the efficacy of the low intensity coherent laser radiation in the treatment of damaged information channels of the optic nerve. The applicant of the embodiments of the present invention has also been investigating the impact of the geometry of the eyeball on the redistribution of the laser energy, and the possibility of monitoring the processes in the eyeball due to the irradiation by an object. There is contradictory evidence in the research works on the impact of the low intensity coherent laser radiation on the retina and the optic nerve. Thus, a well-known American author M. Young argues that Class II lasers that emit approximately 1 mW when aimed directly into the eye with the pupil diameter of 8 mm may cause the retinal irradiance as much as 100 mWcm⁻², and with a 2 mm pupil diameter and with a 2 mm beam diameter if the wavelength of the laser is 633 nm, the radius of the beam waist on the retina is about 1 kWcm⁻², that is 200 times more than the irradiance brought about by the sun.³ At the same time helium-neon lasers have been used for many years to treat retinal and optic nerve disorders (V. V. Okovitov, I. N. Sosin). The applicant has been using 1 mW power helium-neon lasers when treating neuro-ophthalmological disorders for over twenty years The laser therapy was given to 9820 patients ranging from 6 months old to 90 years old. In 8347 Patients both eyes were treated With laser, 1 n 1473 one eye was treated. This clinical practice does not support M. Young's theoretical findings. M. Young's mistake may have occured because his calculations were based on the results of a stationary optic system However an eyeball including the lens, the vitreous body and the ganglion cells of the retina is a dynamic biological system. The applicant proved that M. Young's findings were not reliable and laser therapy can be even used for treating neuro-ophthalmogical disorders in children as young as 6 months old. ³M. Young, Optics and Lasers, Including Fibers and Optical Waveguides, Springer, 2000, pp 235-236.

V. Okovitov believes that threshold levels of laser irradiation which doesn't cause damage to the eye are those with a primary density from 0.5 mW/cm² to 6.4 mW/cm². After the laser treatment one common feature was noted, namely, both microscopic and citochemical changes in the cells were found at a considerable distance (4-6 mm) from the focal spot, that is the reaction of retinal cells to the irradiation was manifested in a large area. The retina reacted as a single functional structure.

This specific reaction of the retina to laser irradiation served as a basis for developing and further use in the clinical practice of laser treatment of some kinds of macular degeneration, amblyopia, and optic nerve atrophy. It is important to note that this research enabled the determination of the threshold levels of laser irradiation which didn't damage the retina.⁴ ⁴V. V. Okovitov, Methods of Physical Therapy in Ophthalmology, Moscow, 1999, pp 90-91.

I. N. Sosin et al. describe a method of laser therapy in ophthalmology. The irradiation is conducted at a distance of 50 cm from the body surface or through a light pipe. The density of the power flow varies from 0.1 to 2-3 mW/cm², the time of the procedure varies from a few seconds to 3 minutes. The overall time of exposure should not exceed 15 minutes. The treatment is conducted daily or every other day. The course of treatment may comprise 1-2 to 10-15 procedures.⁵ ⁵I. N. Sosin, A. G. Buyavikh, Physical Therapy of Eye Disorders, Simferopol, Tavria, 1998, p 83.

There are other methods of laser simulation of the optic nerve. Thus, Linnik et al. use the direct laser stimulation of the optic nerve by means of an orbital puncture and visual control (orbitoscopy). The free end of the photoelectrode is fixed to the skin of the face by sutures. The treatment is conducted by helium-neon laser with the help of the apparatus for the direct laser stimulation of the optic nerve “Lasso.” The output power at the optic generator is 1.5 mW, and the output power at the photoelectrode is 0.4-1.0 mW. The impulse mode is from 1 to 1000 Hz. The duration of one treatment is 40 min. A course of 10 treatments is recommended.⁶ ⁶L. F. Linnik et al., Clinico-Functional Result of Simultaneous Combination Electric and Laser Stimulation of Optic Nerve. IRCTC “Eye Microsurgery”, Moscow//Ophthalmosurgery. Theoretical and Applied Research Journal-No2.-Moscow, 1995.-pp. 44.

The method suggested by L. F. Linnik is not safe as it may cause side effects such as brining in infections and the immune system response to damage tissues. As opposed to this method laser irradiation goes through afferent optic nerve fibers and can be performed through the pupil, which does not cause any side effects described above. L. F. Linnik's method can be used only in patients with completer optic nerve atrophy.

During many years doubts have been growing concerning a hypothesis that information transfer through the optic nerve occurs in the form of electric impulses. The fact that the retina, when illuminated, generates electrical potentials has been known for over a hundred years. Electroretinogram, the recording of these potentials, has proved to be useful for both clinical and research work.⁷ ⁷G. Somjen, Sensory Coding in the Mammalian Nervous System, Moscow, “Mir” 1975, p. 161.

Further research in field showed that laser radiation of low emission intensity enhances the proliferative activity of cells in the tissue culture, increases phagocytal and mitotic activity. Citochemical and radioautographic research showed that extramitotic synthesis of DNA is activated in retinal; ganglion and bipolar cells when the used energy is 20-30% lower than the threshold level. The first signs of the increased synthesis of DNA were observed already a few hours after the exposure to radiation. By the end of 24 hours, the extramitotic synthesis of DNA reached the maximum level followed by a decrease in the next few days, reaching the initial level by the end of the week. The content of DNA in the cell nuclei during this period increased by 25-30%. The reaction of the retinal cells to laser irradiation was not limited by the activation of DNA synthesis as at the same time the synthesis of RNA was observed. This reaction reaches its maximum level 24-48 hours after the exposure.

The described research notes one common feature, namely, both electron microscopic and citochemical changes in the cells were found at a considerable (4-6 mm) distance from the focal spot, that is the reaction of retinal cells to the irradiation was manifested in a large spot, the retina reacted as a single functional structure.

This specific reaction of the retina to laser irradiation served as a basis for developing and further use in the clinical practice of laser treatment of some kinds of macular degeneration and amblyopia.

It is important to note that this research enabled the determination of the threshold levels of laser irradiation which didn't damage the retina.⁸ ⁸V. V. Okovitov, Methods of Physical Therapy in Ophthalmology, Moscow, 1999, p 90-91.

The applicant of the embodiments of the present invention suggested that in this case there might be a possibility of extraneous electrical noises distorting visual signals. In order to study the impact of electromagnetic fields on the distortion of vision, two people were selected. They were suffering from deafening noise in the ears (boomy sound) while being in the areas of radar stations. The applicant of the embodiments of the present invention and the two patients stayed in the area of the radar stations. The patients experienced the deafening noise in the ears, which caused vegetative reactions and a sense of anxiety. After leaving the impacting area, the noise in the ears stopped. The patients didn't notice any changes in vision. This response in the patients can be explained by the impact of acoustic microwaves.

The second doubt was caused by the fact that electrical channels of information transfer can't provide high carrying capacity, which would diminish the reliability of the visual system.

And the third doubt concerns the speed of information transfer, which is one of the important functions for the brain and safety of a person. According to D. Hubel, an impulse spreads along the fiber at a rate of 0.1 to 10 or so meters per second.⁹ Light is a form of an E-field irradiation and spreads in the form of photons or waves in space at the speed of 3×10⁸ m/sec. That is why in order to provide a highly reliable and safe visual system information transfer should be conducted by an optical way, i.e., it should be spread through the fibers of the optic nerve as through a fiberoptic cable. ⁹D. H. Hubel, Eye, Brain, and Vision, New York, 1988, p 18.

(2) The Methodology of Laser Treatment Summarized

The patient is in a sitting or lying position¹⁰. The laser treatment comprises a few manipulations to the damaged area, to the supra- and infra-orbital exit points of the trigeminal nerve and the middle physiological fold of the upper lid when the palpebral fissure is closed, to corporal and auricular points the irradiation is produced by the focused laser beam to the cornea surface. ¹⁰I. N. Sosin, A. G. Buyavikh, Physical Therapy of Eye Disorders, Simferopol, Tavria, 1998, p 83.

The irradiation is conducted at a distance of 50 cm from the body surface or through a light pipe. The density of the power flow varies from 0.1 to 2-3 mW/cm², the time of the procedure for one field (point) from a few seconds to 3 minutes up to 4 points (fields) can be treated during one procedure. The overall time of exposure should not exceed 15 minutes. The treatment is conducted daily or every other day. The course of treatment may comprise from 1-2 to 10-15 procedures. The follow-up treatment can be conducted in 3 or 4 weeks.

(3) Direct Laser Stimulation of the Optic Nerve Summarized

This method is used for treating partial optic nerve atrophy of vascular, post-inflammatory and post-traumatic origin. The method was developed in a research center¹¹ and is conducted as follows: ¹¹L. F. Linnik et al., Clinico-Functional Result of Simultaneous Combination Electric and Laser Stimulation of Optic Nerve. IRCTC “Eye Microsurgery”, Moscow//Ophthalmosurgery. Theoretical and Applied Research Journal-No2.-Moscow, 1995.-pp. 42-47.
A photoelectrode is connected to the optic nerve, with the help of orbital puncture and visual control (orbitoscopy).

The free end of the photoelectrode is fixed to the skin of the face by sutures.
The treatment is conducted by a helium-neon laser, with the help of the apparatus for the direct laser stimulation of the optic nerve “Lasso.”
The output power at the optic generator is 1.5 mW.
The output power at the photoelectrode is 0.4-1.0 mW.
The impulse mode is from 1 to 1000 Hz.
The duration of one treatment is 40 minutes.
A course of 10 treatments is recommended¹². ¹²I. N. Sosin, A. G. Buyavikh, Physical Therapy of Eye Disorders, Simferopol, Tavria, 1998, p 98.

(4) Relevant Prior Art

Numerous innovations for eye treatments have been provided in the prior art that will be described below in chronological order to show advancement in the art, and which is incorporated herein by reference thereto. Even though these innovations may be suitable for the specific individual purposes to which they address, however, they differ from the present invention in that they do not teach a method for experimentally optic transmitting information through an optic nerve.

(a) U.S. Pat. No. 6,313,451 B1 to Streeter.

U.S. Pat. No. 6,313,451 B1 issued to Streeter on Nov. 6, 2001 in U.S. class 607 and subclass 89 teaches a low level laser therapy apparatus for treatment of various tissue injuries. In one embodiment, the apparatus includes a handheld laser probe coupled to a control unit for selecting and controlling laser energy dosage from about 1 joule/point to about 10 joules/point. The apparatus emits laser energy at a wavelength from about 630 nm to about 904 nm, with a mean power output of between about 100 mW to about 500 mW. The apparatus further includes an access control mechanism to limit operability to trained personnel.

(b) U.S. Pat. No. 6,319,274 B1 to Shadduck.

U.S. Pat. No. 6,319,274 B1 issued to Shadduck on Nov. 20, 2001 in U.S. class 607 and subclass 89 teaches an apparatus and technique for transscleral light-mediated biostimulation of the trabecular plates of a patient's eye in a treatment for glaucoma or ocular hypertension. The apparatus includes: a working end geometry for contacting the anterior surface of the sclera and cornea to insure that a laser emission reaches the trabecular meshwork from a particular location on the anterior surface of the sclera; a laser energy source providing a wavelength appropriate for absorption beneath the anterior scleral surface to the depth of the trabecular plates; and a dosimetry control system for controlling the exposure of the laser emission at the particular spatial locations. The device uses a light energy source that emits wavelengths in the near-infrared portion of the spectrum, preferably in the range of about 1.30 μm to 1.40 μm or from about 1.55 μm to 1.85 μm. The depth of absorption of such wavelength ranges will extend through most, if not all, of the thickness of the sclera (750 μm to 950 μm). In accordance with a proposed method of trabecular biostimulation, the targeted region is elevated in temperature to a range between about 40° C. to 55° C. for a period of time ranging from about 1 second to 120 seconds or more.

(c) U.S. Pat. No. 6,471,691 B1 to Kobayashi et al.

U.S. Pat. No. 6,471,691 B1 issued to Kobayashi et al. on Oct. 29, 2002 in U.S. class 606 and subclass 4 teaches an ophthalmic treatment apparatus using therapy and diagnostic laser light sources whose beams are deflected two-dimensionally via an optical deflector and a galvanomirror and directed on the eye fundus to produce a fundus image on a display monitor. A photosensitive substance that accumulates specifically in neovascular regions is administered to the patient to define the region where the neovascular tissues are located. When the region is to be treated, the therapy laser light source is activated and its intensity is amplified by a controller and a driver for driving a light modulator. This arrangement assures a reliable definition of the affected region and enables only the neovascular tissues to be destroyed or sealed off because the laser intensity can be amplified at the region concerned.

(d) U.S. Pat. No. 6,524,330 B1 to Khoobehi et al.

U.S. Pat. No. 6,524,330 B1 issued to Khoobehi et al. on Feb. 25, 2003 in U.S. class 607 and subclass 89 teaches a method for treating abnormal blood vessel growth and proliferation. Members of the hypocrellin class of compounds, such as hypocrellin A, hypocrellin B, and/or amino-substituted derivatives of hypocrellin B are administered and photoactivated with photodynamic therapy. The method may be used, for example, to treat ocular blood vessel proliferation as occurs with macular degeneration.

(e) U.S. Patent Application Publication Number US2005/0065577 A1 to McArthur et al.

United States Patent Application Publication Number US 2005/0065577 A1 published to McArthur et al. on Mar. 24, 2005 in U.S. class 607 and subclass 88 teaches a method for the therapeutic treatment of biological tissue of a patient with a low level laser. The method is achieved by diagnosing the nature and extent of the tissue disorder, establishing at least one treatment area, exposing the treatment area to monochromatic coherent light below the level necessary to cause thermal damage to the tissue being treated, and treating the treatment area for sufficient treatment time to produce clinically beneficial effects by delivering a dosage greater than 20 joules/cm². The light is in the near infrared portion of the electromagnetic spectrum.

(f) U.S. Pat. No. 6,942,655 B2 to Peyman.

U.S. Pat. No. 6,942,655 B2 issued to Peyman on Sep. 13, 2005 in U.S. class 606 and subclass 4 teaches that age-related macular degeneration (AMD) results in the formation of new blood vessels in the eye. The walls of these vessels leak fluid, which causes scarring in the surrounding tissue and results in reduced vision or loss of vision. Photodynamic therapy (PDT) alone has been used to treat AMD, but many retreatments are needed, which cause further damage to the already diseased area. Laser treatment to coagulate the fluid actually causes additional new vessels to form. The method of treating patients with both PDT and scatter threshold laser coagulation therapy surprisingly either improved vision, or prevented further loss of vision. Moreover, the combined treatment eliminated the need for retreatment, and did not generate new vessel growth. Threshold laser coagulation and PDT may be administered within the same treatment session or either may be administered first and the other may be administered within ninety days.

(g) U.S. Patent Application Publication Number US 2006/0184214 A1 to McDaniel.

United States Patent Application Publication Number US 2006/0184214 A1 published to McDaniel on Aug. 17, 2006 in U.S. class 607 and subclass 89 teaches a system and method for treatment of cells and, in particular, visual pathway disorders. More particularly, the system and method are directed toward the photomodulation and/or photorejuvenation of retinal epithelial cells, to treat a variety of vision disorders. The process of treating retinal cells to reduce or reverse the effects of visual pathway disorders employs a narrowband source of multichromatic light applied to the retinal cells to deliver a very low energy fluence.

(h) U.S. Patent Application Publication Number US 2007/0060984 A1 to Webb et al.

United States Patent Application Publication Number US 2007/0060984 A1 published to Webb et al. on Mar. 15, 2007 in U.S. class 607 and subclass 89 teaches a nerve-stimulation device and method using light to provide a source of precise stimulation on one or more nerve fibers. In some embodiments, this simulation is provided through a device and method wherein a laser- or LED-light-generating source is operatively coupled to an optical fiber, which in turn is coupled to a plug in the end of a holder in a sheath. Light is then passed from the light source through the optical fiber to the holder and out a selected optical tip on the sheath to provide an efficacious amount of light to simulate nerves. In some embodiments, the device is constructed from non-magnetic material, such as glass, plastic, or ceramics. In some embodiments, the light emanating from the optical tip can be controlled manually or automatically. Some embodiments omit the fiber and use light directly from the laser diode.

It is apparent that numerous innovations for eye treatments have been provided in the prior art that are adapted to be used. Furthermore, even though these innovations may be suitable for the specific individual purposes to which they address, however, they would not be suitable for the purposes of the embodiments of the present invention as heretofore described, namely, a method for experimentally optic transmitting information through an optic nerve.

### SUMMARY OF THE INVENTION

Thus, an object of the embodiments of the present invention is to provide a method for experimentally optic transmitting information through an optic nerve, which avoids the disadvantages of the prior art.

Briefly stated, another object of the embodiments of the present invention is to provide a method for experimentally optic transmitting information through an optic nerve. The method includes the steps of transecting the optic nerve at the beginning of the optic chiasm in order to check the propagation of a laser beam along efferent fibers of the optic nerve, putting a metal screen on the optic nerve, fixing the metal screen at the exit of the optic nerve from the eyeball, directing the laser beam at the butt end of the optic nerve, observing visible luminescence of the optic nerve with holographic effect, projecting the laser beam in the pupil, and observing visible luminescence of the pupil.

The novel features considered characteristic of the embodiments of the present invention are set forth in the appended claims. The embodiments of the present invention themselves, however, both as to their construction and their method of operation together with additional objects and advantages thereof will be best understood from the following description of the specific embodiments when read and understood in connection with the accompanying drawing.

### BRIEF DESCRIPTION OF THE DRAWING

The figures of the drawing are briefly described as follows:

FIG. 1 is a diagrammatic top plan view, with parts broken away, demonstrating that when laser beams are S shined through dilated pupils, uniform luminesce of the eye balls and optic nerves are observable in contradistinction to a pinhole effect on the retinas;

FIG. 2 is a diagrammatic top plan view, with parts broken away, of the optic nerves transected behind the optic chiasm, with laser beams aimed into the transections towards the eye balls, demonstrating that luminescence of the optic chiasm and the optic nerves was not observable;

FIG. 3 is a diagrammatic top plan view, with parts broken away, of the optic nerves transected at the beginning of the optic chiasm, with laser beams aimed into the transections towards the eyeballs, demonstrating that luminescence of the optic nerves with holographic effect, and the emission of laser beams in the pupils was observed;

FIG. 4 is a diagrammatic side elevational view, with parts broken away, demonstrating that a laser beam shined in the area of the visual cortex, through the occipital bone, is visibly observable 5 mm deep into the striate cortex; and

FIG. 5 is a diagrammatic side elevational view, with parts broken away, demonstrating a laser beam shined in the area of the visual cortex with the occipital bone.

### LIST OF REFERENCE NUMERALS UTILIZED IN DRAWING

10 luminescence 10 of optic nerve 12
12 optic nerve
14 optic chiasm
16 optic tract of lateral geniculate body 17
17 lateral geniculate body
18 laser beam
20 metal screen
22 exit of optic nerve 12
24 eyeball
26 butt end of optic nerve 12
28 pupil
29 luminescence of pupil 28
30 visual cortex/striate cortex
32 occipital bone
34 luminescence of striate cortex 30

### DETAILED DESCRIPTION OF PREFERRED EMBODIMENT

A. Pre-Experiment

Before conducting the experiment, the applicant of the embodiments of the present invention did an autopsy on ten bodies to determine the average length of the optic nerve from the optic chiasm, which varied from 35 to 55 mm, with an average diameter of 4 mm. The authors of the Clinical Neuro-Ophthalmology give another figure for the length of the optic nerve, namely, 40-50 mm.¹³ ³N. R. Miller et al., Walsh & Hoyt's Clinical Neuro-Ophthalmology, Lippincot Williams & Wilkins, 2003, p 25.

B. The Experiment

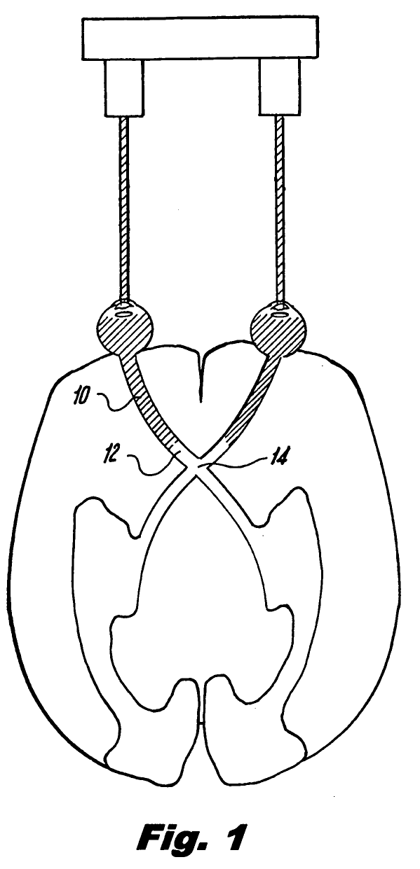

In order to verify, the applicant of the embodiments of the present invention developed a method, the first step of which was to check the redistribution of the laser irradiation in the eyeball. With this purpose, a helium-neon laser with output power of 1 mW and a beam diameter of 2 mm was used. The experiment was conducted on the body of a forty-five-year old man, which was delivered to the morgue 40 minutes after the fact of his death was verified. During the autopsy, the eyeball, the optic nerve, the optic chiasm, and the lateral geniculate body were extracted. Through a dilated pupil, 6 mm in diameter, the laser beam was directed into the eyeball. The inventor observed not a pinhole impact on the retina but uniform luminescence of the eye membrane, which penetrated even through the membrane. As shown in FIG. 1, which is a diagrammatic top plan view, with parts broken away, demonstrating that when laser beams are shined through dilated pupils, uniform luminesce of the eye balls and optic nerves are observable in contradistinction to a pinhole effect on the retinas, there was also a visible luminescence 10 of the optic nerve 12, with the holographic effect which faded away at a distance of 4 mm from the optic chiasm 14.

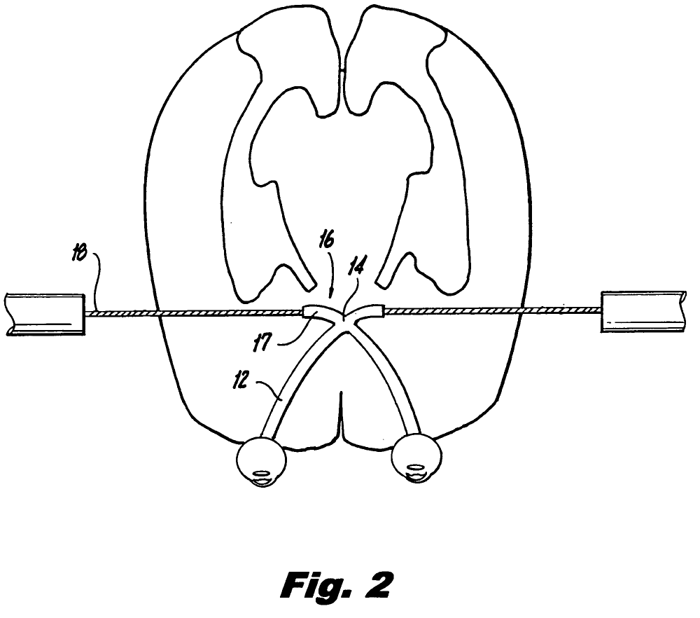

As shown in FIG. 2, which is a diagrammatic top plan view, with parts broken away, of the optic nerves transected behind the optic chiasm, with laser beams aimed into the transections towards the eye balls, demonstrating that luminescence of the optic chiasm and the optic nerves was not observable, after the transection of the optic tract 16 of the lateral geniculate body 17 behind the optic chiasm 14, the laser beam 18 was directed at the optic chiasm 14. The luminescence of the optic chiasm 14 and the optic nerve 12 was not observed.

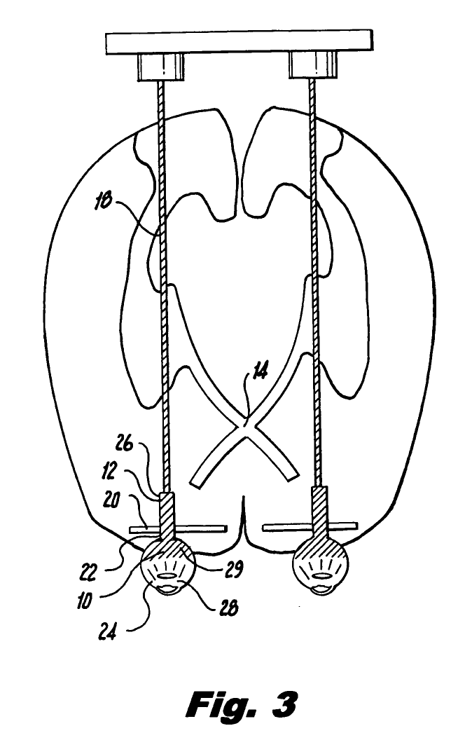

As shown in FIG. 3, which is a diagrammatic top plan view, with parts broken away, of the optic nerves transected at the beginning of the optic chiasm, with laser beams aimed into the transections towards the eyeballs, demonstrating that luminescence of the optic nerves with holographic effect, and the emission of laser beams in the pupils was observed, in order to check the propagation of the laser irradiation along the efferent fibers of the optic nerve 12, the optic nerve 12 was transected at the beginning of the optic chiasm 14. A metal screen 20 was put on the optic nerve 12. It was fixed at the exit 22 of the optic nerve 12 from the eyeball 24. When the laser beam 18 was directed at the butt end 26 of the optic nerve 12, the applicant of the embodiments of the present invention observed visible luminescence 10 of the optic nerve 12 with holographic effect, and the projection of the laser irradiation in the pupil 28, that is, the luminescence 29 of the pupil 28 was also observed.

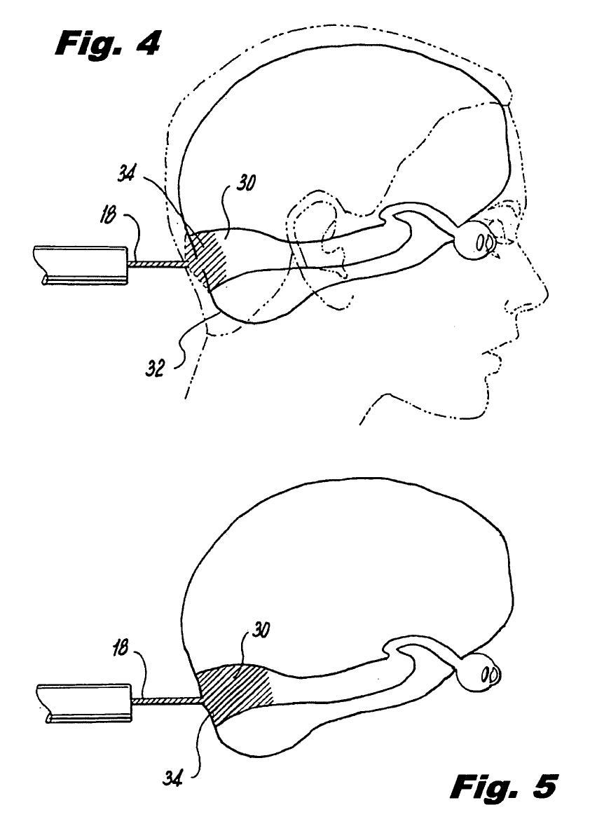

As shown in FIG. 4, which is a diagrammatic side elevational view, with parts broken away, demonstrating that a laser beam shined in the area of the visual cortex, through the occipital bone, is visibly observable 5 mm deep into the striate cortex, while investigating the proliferation of the laser irradiation 18 in the area of the visual cortex 30 through the occipital bone 32, a visible luminescence 34 5 mm deep into the striate cortex 30 was observed.

And when the laser irradiation 18 had a direct impact on the striate cortex 30, as shown in FIG. 5, which is a diagrammatic side elevational view, with parts broken away, demonstrating that a laser beam shined in the area of the visual cortex is visibly observable 10 mm deep into the striate cortex, the visible luminescence 34 was observed at a depth of 10 mm. 3.5 hours after the beginning of the experiment, an obvious dimness of the cornea was noticeable. The luminescence of the optic nerve 12 ceased. When the laser beam 18 was directed at the peripheral nerve nerva ulnaris, there was no luminescence.

This experiment shows that when light impacts the retina, photochemical processes are set off, by which atoms and molecules are excited or ionized.

When a photon is absorbed by the rhodopsin complex, the molecules become excited. When they return to the normal state, they emit luminescent irradiation. The term “luminescence” is used to describe the emission of light for both luminescence and fluorescence. An atom can be viewed as a tiny resonator, which is able to irradiate or absorb electromagnetic waves.

Under the influence of photons, the molecular processes of the transformation of light energy into chemical energy take place. Chemical sensors are connected with an optic way of signal transformation. The transfer and processing of information occur concurrently and through individual channels.

For image transfer, the retina uses an information capacity of ten million bits per second. The speed of subject perception is only a few scores of bits per second. It's almost six orders lower.

The retina fulfills a complex task. It transforms optic signals into electrical ones, separates signals from noise, and again transfers them through the fibers of the optic nerve as optic signals. Having passed through the fibers of the optic nerve, the signal becomes weaker. The most common linear effect is signal attenuation, which is weakening of a signal while it passes through the fibers of the optic nerve. The molecules, which can act as luminescent centers, have a complex structure. The exact distribution of charges in the center of irradiation, and its changes in the state of excitation, are not yet known. Experience shows that the behavior of different radiators at a first approximation is described on the basis of simplified models of electric and magnetic dipoles, and also a quadrupole. The oscillation of the charges of the electric dipole is accompanied by the emission of a light quantum.

The initial impact of the light energy sets off a fast reaction on the background of lengthy molecular transformations of the rhodopsin, the molecular restoration of which is accompanied by light emission.

For the signals to be able to transfer information, they should be modulated. In the retina there are optic and chemical centers. Chemical sensors are connected with the optic transfer of signals. In the retina, the amplitude manipulation of signals, which is connected with on- and off-centers, takes place. The signals are either present (1) or absent (0).

C. The Conclusions

It will be understood that each of the elements described above or two or more together may also find a useful application in other types of constructions differing from the types described above.

While the embodiments of the present invention have been illustrated and described as embodied in a method for experimentally optic transmitting information through an optic nerve, however, they are not limited to the details shown, since it will be understood that various omissions, modifications, substitutions, and changes in the forms and details of the embodiments of the present invention illustrated and their operation can be made by those skilled in the art without departing in any way from the spirit of the embodiments of the present invention.

Without further analysis the foregoing will so fully reveal the gist of the embodiments of the present invention that others can by applying current knowledge readily adapt them for various applications without omitting features that from the standpoint of prior art fairly constitute characteristics of the generic or specific aspects of the embodiments of the present invention.

The invention claimed is:

1. A method for experimentally optic transmitting information through an optic nerve, comprising the steps of:
a) projecting a laser beam through the pupil, the vitreous body, the ganglion cells of the retina and the afferent nerve fibers to the beginning of the optic chiasm;
b) observing visible luminescence of the eyeball and afferent nerve fibers to the beginning of the optic chiasm with holographic effect;
c) transecting the optic nerve at the beginning of the optic chiasm in order to check the propagation of a laser beam along efferent fibers of the optic nerve;
d) protecting the eyeball with a metal screen;
e) fixing the metal screen at the exit of the optic nerve from the eyeball;
f) directing the laser beam at the butt end of the optic nerve;
g) observing visible luminescence of the optic nerve with holographic effect;
h) projecting the laser beam in the pupil; and
i) observing visible luminescence of the pupil.
